(19) United States
(12) Patent Application Publication
(10) Pub. No.: US 2010/0170518 A1
(43) Pub. Date: Jul. 8, 2010

(54) CONDOM WITH ONE-WAY SEALING CLOSURE

(76) Inventor: Adam Goldberg, Mercer Island, WA (US)

Correspondence Address: MITCH HARRIS, LLC - GENERAL, P.O. BOX 7998, ATHENS, GA, 30604, US

(21) Appl. No.: 12/349,549

(22) Filed: Jan. 7, 2009

(51) Int. Cl. A61F 6/04

(52) U.S. Cl. 128/844

(57) ABSTRACT

A condom with a one-way sealing closure provides for an irreversible seal that once activated, prevents leakage of the condom contents after coitus, for sample collection or general disposal. The one-way sealing closure is provided by a cord that is disposed around the proximal circumference of the condom, and may be secured in an internal rolled channel or by guides attached to the outside wall of the condom. Additional features that may be included in the condom include various forms of one-way locking closures, visual and tactile guides, and a pull tab for operating the closure.

### BACKGROUND OF THE INVENTION

1. Field of the Invention

The present invention relates to devices for shielding the male penis during sexual intercourse or sample collection, and more particularly a condom closure including a one-way closure.

2. Description of the Related Art

Good hygiene and health practices require that biological waste be handled with care. Condoms are particularly hard to dispose of because it is well known that a thin rubber tubular sheath type of condom occasionally allows seminal fluid to spill. Spillage usually happens during removal or disposal of the condom after ejaculation. The reason for the spillage is the widened mouth of the condom due to coital movement and stretching over the penis. After the removal of the condom, the widened mouthy opening allows ample space for the semen to spill on to the body of the user, the body of another, or surrounding surfaces which mucous membranes of others might later contact.

Various kinds of condoms have been used in the past to tighten the connection or provide a seal between condom and penis as to prevent semen from contacting the user's partner. A re-closable condom, for example, is described in U.S. Pat. No. 4,564,006; one embodiment has a zip closure along the length of the condom in order to attach the condom to a flaccid penis. U.S. Pat. No. 5,803,085 describes a non-elastic condom having a two ended tether or heat resistant elastic band adjacent the insertion opening that secures the sheath securely about the body portion of the penis to maintain the sheath over the penis while in use. Similarly, the condom described in U.S. Pat. No. 5,855,206 discusses a closure that provides a seal at the base of the penis to provide protection from disease and pregnancy resulting from fluid leakage, and if suitably tight, can prolong erections. The numerous potential closures discussed in the above-referenced U.S. patent include buckles, VELCRO, and in one example, an extremely long cord which is attached at one end and wrapped multiple times around the base of the condom.

While the above-described condom closures may be suitable for maintaining a seal during use, a condom designed to have a removable closure at the entry end does not solve the problem of spillage as described above. In fact, a permanent opening is inherent in the design of standard condoms currently available, and is present in each of the condoms described in the above-referenced patents. In condoms that are adapted to ensure connection to an appendage for use as catheters or for other purposes, reversible closing mechanisms, such as buckles and strings, do not provide an effective means for sanitary disposal, since when the closure is released the contents of the condom are also much more likely to spill. Standard condoms are not intended to provide a sealed enclosure after sample collection or coitus has occurred. Therefore, it is highly desirable to provide a means for safely disposing of and/or transporting a condom and its contents, as well as preserving the contents.

### SUMMARY OF THE INVENTION

The objective of providing a condom that provides a permanent and secure closing mechanism for sample collection/sperm donation transport and for general disposal, is provided in a condom and its method of operation.

The condom includes an elongated generally tubular sheath of thin, flexible material and a closure for sealing the proximal opening in a substantially irreversible manner during and after the condom is removed. A securing mechanism is provided adjacent to the condom's proximal (insertion) opening to irreversibly and securely seal the sheath after removal from the penis. The securing mechanism may include a constriction mechanism, such as a two-ended tether with a bead and loop fastening system or a cord with a series of ridges along its length for engaging a fastener, in order to completely constrict the insertion opening end of the sheath member. Additionally, the sheath may be formed with a reinforced rim at the proximal opening and may include visual and/or tactile indicators as to the position of the constriction mechanism.

The foregoing and other objectives, features, and advantages of the invention will be apparent from the following, more particular description of the preferred embodiment of the invention, as illustrated in the accompanying drawings.

### BRIEF DESCRIPTION OF DRAWINGS

The novel features believed characteristic of the invention are set forth in the appended claims. The invention itself, however, as the mode of use, further objectives, and advantages thereof, will best be understood by reference to the following detailed description of an illustrative embodiment when read in conjunction with the accompanying drawings, wherein like reference materials indicate like materials and:

FIG. 1 is a pictorial view of a condom in accordance with an embodiment of the present invention.

FIG. 2 is a detail view of the condom of FIG. 1 and FIG. 3 showing features in the vicinity of reinforcement band 13 in accordance with an embodiment of the present invention.

FIG. 3 is a pictorial view of a condom in accordance with an alternative embodiment of the present invention.

FIG. 4 is a pictorial view of a proximal end of a condom including color coded, touch-perceptible projections 30 as may be provided on the condoms of FIG. 1 and FIG. 3 in accordance with an embodiment of the present invention.

FIGS. 5A, 5B, and 5C are illustrations of various one-way locking drawstrings that may be included within the condoms of FIG. 1 and FIG. 3 in accordance with embodiments of the present invention.

### DESCRIPTION OF ILLUSTRATIVE EMBODIMENT

The present invention encompasses a condom that provides for a one-way closure that is substantially irreversible. The condom permanently seals at the proximal end, avoiding spillage of biological waste for prevention of disease and/or preventing contamination of samples. In describing a permanent seal, it is not indicated that the condom cannot be re-opened, only that such operation would be difficult to perform manually (i.e., without cutters or the like) and that accidental reversal of the closure is prevented by the one-way closure mechanisms described below.

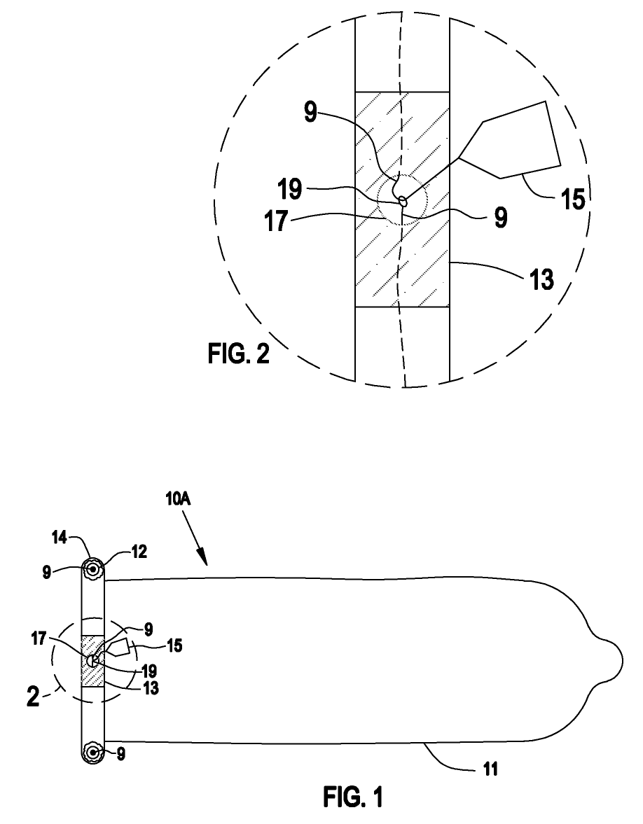

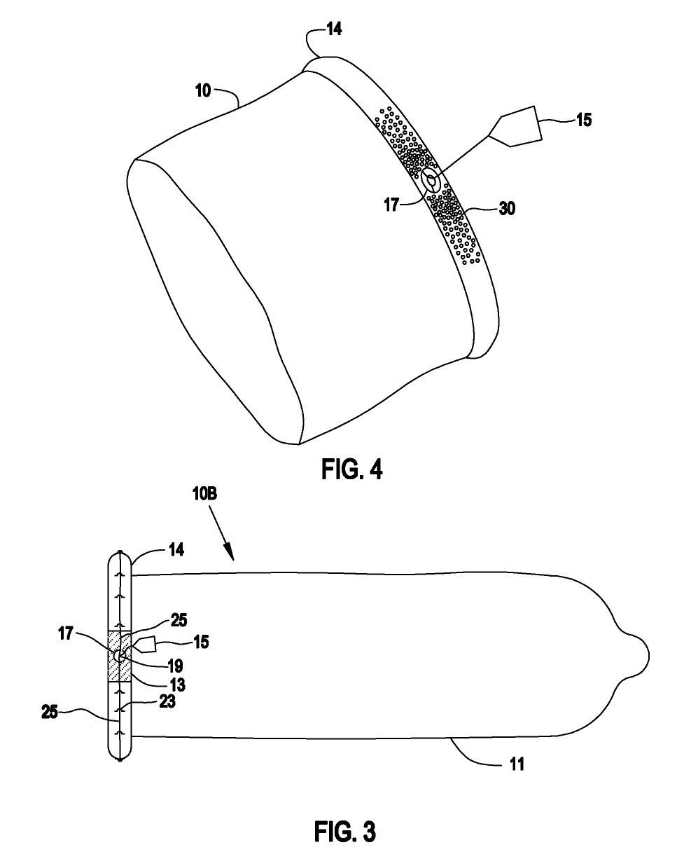

Referring now to FIG. 1, a completely unrolled condom 10A in accordance with an embodiment of the invention is illustrated. Condom 10A includes a hollow and tubular sheath 11 made of elastic material and having a closed distal end. Condom 10A further includes an open, proximal end 14 into which a penis is inserted during use. A sealing mechanism is provided in condom 10A, which seals off proximal end 14 and completely encloses the contents of condom 10A, after the sealing mechanism has been operated. The mechanism for sealing may be a corded closure 9 used as a drawstring as shown. A corded closure 9, made of a material of reliable strength, extends around the periphery of proximal end 14 of the condom in FIG. 1 and FIG. 3. Corded closure 9 may be attached by rolling the proximal end 14 of tubular sheath 11 over corded closure 9 during manufacture, so that corded closure 9 is captured within a channel 12 formed by the rolling. Corded closure 9 may be formed from a compressible material or padded and/or lubrication may be provided within channel 12, to reduce the likelihood of tearing of a drawstring aperture 17 through which corded closure 9 extends from channel 12. Channel 12 may be completed by folding over and securing an edge of proximal end 14 of tubular sheath 11 using glue or another suitable adhesive. Alternatively, corded closure 9 can be inserted through a pre-formed channel 12, formed during the manufacture of condom 10A. If either method is used, a grasping tab 15 may be attached to corded closure 9 after insertion or rolling of tubular sheath 11 to simplify insertion of corded closure 9 into a pre-formed channel 12.

In order to tighten corded closure 9 about proximal end 14, grasping tab 15 is attached to an end of corded closure 9 and may be pulled from the vicinity of drawstring aperture 17. Pulling on grasping tab 15 tightens corded closure 9 within channel 12, closing the circumference of proximal end 14 of condom 10A, effectively sealing condom 10A, so that any fluids present on the inside of condom 10A are retained without spillage. Tab 15 may be coated with or fabricated using a luminescent material, so that tab 15 is visible even under poor lighting conditions.

A loop 19 may be preformed on corded closure 9 and corded closure 9 inserted therethrough prior to attachment of pull tab 15. Alternatively, tab 15 may be pre-attached and corded closure 9 looped around itself and secured by adhesive, welding, or other suitable means. In another alternative embodiment, loop 19 may be a pre-formed ring-shaped structure that is bonded to the end of corded closure 9. Corded closure 9 is passed through loop 19 at the end of corded closure opposing grasping tab 15 so that the position of loop 19 advances along corded closure 9 when grasping tab 15 is pulled, until the inner surface of sheath 11 closes at proximal end 14 of condom 10A. The resulting closure providing a substantially irreversible and complete seal so that the user can safely transport or dispose of the biological material contained therein. The diameter of loop 19 should be sufficiently small, or the material of corded closure 9 should have a surface friction characteristic, such as a friction-activated adhesive coating, which ensures that the closure does not re-open after tightening. Alternatively, corded closure 9 can be provided with mechanical locking features, as will be described in further detail below. Tab 15 facilitates tightening corded closure 9 to effect closure as well as preventing the first end of drawstring from passing through loop 19, preventing detachment of corded closure 9 from loop 19 prior to use.

Referring now to FIG. 2, details of condom 10A in accordance with an embodiment of the present invention are shown. A reinforcement band 13 (represented by the shaded region) is applied or formed around drawstring aperture 17 of the present invention, to strengthen the material forming channel 12 against tearing when corded closure 9 is tightened. As shown, reinforcement band 13 may be formed by additional material or panels applied around drawstring aperture 17.

Referring now to FIG. 3, a completely unrolled condom 10B in accordance with another embodiment of the present invention is shown. Condom 10B is similar to condom 10A, and therefore only differences between them will be described below. In the depicted embodiment, corded closure 9 is movably secured by guides 23 disposed around the circumference of the outer wall of proximal end 14 of the condom 10B. Guides 23 can be formed on the surface of the condom during manufacture or attached by adhesive or other suitable bonding method. Proximal end 14 is generally rolled over the guides after manufacture to conceal and prevent contact with guides 23 during use. Therefore, main sheath 11 has a length sufficient to prevent at least the last turn from requiring unrolling during use of condom 10B. Pulling on grasping tab 15 tightens corded closure 9, closing the circumference of proximal end 14 of condom 10B, effectively sealing condom 10B, so that any fluids present on the inside of condom 10B are retained without spillage.

Referring now to FIG. 4, details of a condom 10 in accordance with another embodiment of the present invention is shown, and such features may be incorporated in either condom 10A or condom 10B as described above. Touch-perceptible projections 30 are integrated along proximal end 14 to indicate to the user the proximity of the drawstring aperture 17 and grasping tab 15, facilitating operation of the sealing mechanism in poor lighting conditions or without requiring the person sealing the condom to be able to view grasping tab 15. Projections 30 are color-coded in the illustrated embodiment, and a color-coding scheme may be used alone (i.e., without tactile projections) when the ability to view the closing operation is expected. The color of projections 30 may vary in hue, intensity or both, providing easy visual observation of the location of aperture 17 and the expected location of grasping tab 15.

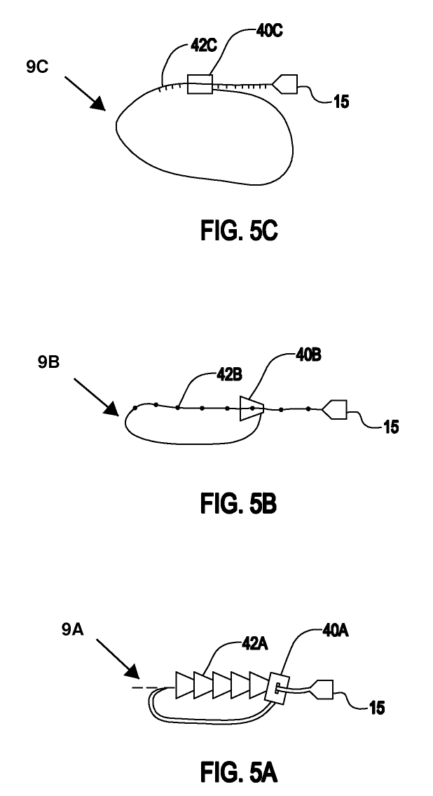

Referring now to FIGS. 5A-5C, various locking closures 9A-9C as may be used to implement corded closure 9 in condoms 10A of FIG. 1 and condom 10B of FIG. 3 are illustrated in accordance with embodiments of the present invention. It is understood that when such locking closures are used in combination with guides 23 of FIG. 3, locking closures 9A-9C are sized so as to pass through the guides sufficiently freely to effect closure when grasping tab 15 is pulled. Referring now to FIG. 5A, a corded closure 9A suitable for use in condom 10A of FIG. 1 and condom 10B of FIG. 3 has a saw-tooth shaped profile 42A along the length of corded closure 9A and further include a locking receptacle 40A terminating one end. Upon cinching corded closure 9A, lock retention teeth 42A engage locking receptacle 40A thereby locking corded closure 9A and preventing reversal, allowing corded closure 9A to be tightened, but not loosened.

Referring now to FIG. 5B, another corded closure 9B suitable for use in condom 10A of FIG. 1 and condom 10B of FIG. 3 has spherical projections 42B disposed along the length of corded closure 9B and also includes a locking receptacle 40B terminating one end. Upon cinching corded closure 9B, lock retention spherical projections 42B engage locking receptacle 40B thereby locking corded closure 9B and preventing reversal, allowing corded closure 9B to be tightened, but not loosened.

Referring now to FIG. 5C, yet another a corded closure 9C suitable for use in condom 10A of FIG. 1 and condom 10B of FIG. 3 has a series of ridges 42C disposed along the length of corded closure 9C and also includes a locking receptacle 40C terminal terminating one end. Upon cinching corded closure 9C, ridges 42C engage locking receptacle 40C thereby locking corded closure 9C and preventing reversal, allowing corded closure 9C to be tightened, but not loosened.

While the invention has been particularly shown and described with reference to the preferred embodiments thereof, it will be understood by those skilled in the art that the foregoing and other changes in form, and details may be made therein without departing from the spirit and scope of the invention.

What is claimed is:

1. A condom for placement over penis, comprising:
a substantially tubular sheath of elastic material extending along a longitudinal axis, that is closed at a distal end of the condom and forming a proximal opening at a proximal end of the condom; and
a one-way sealing closure for closing the proximal opening after use of the condom to provide a substantially liquid-tight seal.

2. The condom of claim 1, wherein the one-way sealing closure comprises a corded closure secured at the proximal end of the condom and extending around a circumference of the sheath at the proximal end thereof.

3. The condom of claim 2, wherein the corded closure is a locking corded closure including one or more features for preventing reopening of the liquid-tight seal.

4. The condom of claim 3, wherein the one-way locking corded closure comprises:
a plurality of protrusions disposed along a length of the corded closure; and
a locking receptacle disposed at a first end of the corded closure and for receiving a second end of the corded closure and permitting the protrusions to pass freely in one direction when pulled during sealing of the proximal opening.

5. The condom of claim 2, wherein the one-way locking corded closure comprises:
a drawstring; and
a loop disposed at a first end of the drawstring for receiving a second end of the drawstring therethrough.

6. The condom of claim 2, wherein the corded closure is disposed in at least one channel located at the proximal end of the condom for guiding the corded closure, wherein the channel extends substantially around the circumference of a proximal end of the sheath.

7. The condom of claim 6, wherein the channel is formed by a rolled tube formed by a proximal end of the sheath, wherein an inner surface of the tube is substantially contiguous with an outer surface of the sheath and the corded closure extends through an aperture disposed on outer surface of the rolled tube and passes out of the channel therethrough.

8. The condom of claim 7, further comprising a reinforcement band disposed around the aperture and extending circumferentially around the sheath.

9. The condom of claim 2, further comprising a plurality of guides for guiding the corded closure and disposed on and circumferentially distributed around an outer surface of the proximal end of the condom.

10. The condom of claim 2, further comprising a plurality of projections disposed at the proximal end of the condom for providing tactile information as to the location of an end of the corded closure at the proximal end of the condom.

11. The condom of claim 2, wherein the condom includes a color-coded marking on the proximal end of the condom providing visual information as to the location of the end of the corded closure disposed at the proximal end of the condom.

12. The condom of claim 2, further comprising a pull tab secured to the one-way corded closure.

13. A method of manufacturing a condom, comprising:
forming a substantially tubular sheath from an elastic material, wherein the sheath is closed at a distal end of the condom and open at a proximal end of the condom; and
securing a one-way corded closure to the sheath around a circumference of the proximal end of the condom, wherein the one-way corded closure is movably retained at the proximal end of the condom.

14. The method of claim 13, wherein the securing is performed by inserting the one-way corded closure around the circumference of the sheath at proximal end of the condom.

15. The method of claim 14, wherein the corded closure is a one-way locking closure, and wherein the securing is performed by inserting the one-way corded locking closure around the circumference of the sheath and locking the closure into a loop by activating at least one locking position of the locking closure.

16. The method of claim 13, further comprising:
rolling the proximate end of the sheath to form a tubular channel extending around the circumference of the proximal end of the condom, and wherein the securing secures the one-way corded closure within the tubular channel;
forming an aperture in an outer surface of the tubular channel; and
passing the corded closure through the aperture to extend the corded closure outside of the channel.

17. The method of claim 16, further comprising bonding or gluing the proximal edge of the sheath to the outer surface of the sheath at a location along the sheath away from the proximal edge of the sheath.

18. The method of claim 16 further comprising forming a reinforcement band in the outer surface of the tubular channel and surrounding the aperture.

19. The method of claim 13, wherein the securing is performed by securing the corded one-way closure with a plurality of guides disposed around the periphery of the proximal opening.

20. The method of claim 13, further comprising attaching a grasping tab to an end of the corded closure.

21. A method for sealing a condom, comprising:
pulling an end of a one-way closure located at the proximal end of the condom; and
in response to the pulling, constricting an opening at the proximal end of the condom by tensioning the one-way closure to substantially seal the opening.

22. The method of claim 21, wherein the one-way closure is a one-way corded closure extending around the circumference of the opening to form a loop, and wherein the constricting is performed by closing the loop in response to the pulling.

23. The method of claim 21, wherein the one-way closure is a locking one-way corded closure, and wherein the tensioning further activates one or more locking features disposed along a length of the one-way corded closure.
